(19) United States
(12) Patent Application Publication
(10) Pub. No.: US 2021/0212633 A1
(43) Pub. Date: Jul. 15, 2021

(54) METHODS AND APPARATUS FOR PORTABLE UNIVERSAL MONITORING DEVICE

(71) Applicants: James Ryan, Scottsdale, AZ (US); Tammy Ryan, Scottsdale, AZ (US)

(72) Inventors: James Ryan, Scottsdale, AZ (US); Tammy Ryan, Scottsdale, AZ (US)

(21) Appl. No.: 17/145,711

(22) Filed: Jan. 11, 2021

Related U.S. Application Data
(60) Provisional application No. 62/959,273, filed on Jan. 10, 2020.

(51) Int. Cl. A61B 5/00; H04Q 9/00; H05K 5/00; G01N 33/483

(52) U.S. Cl. CPC A61B 5/681; H04Q 9/00; H05K 5/0086; G01N 33/483; A61B 2560/0443; A61B 2560/0214; A61B 5/002; A61B 5/0022; H04Q 2209/40

(57) ABSTRACT

A portable universal monitoring device according to various aspects of the present technology includes a portable body housing a plurality of sensor devices. The portable body is configured to be repeatedly attached to and detached from one or more objects such as a wristwatch. The portable body is configured to be discretely positioned on the object to remain unseen without negatively affecting the fit, function, operation, or comfort of the object during use. The portable body may be used interchangeably with any number of objects without requiring the objects themselves to be modified.

### CROSS-REFERENCES TO RELATED APPLICATIONS

This application claims the benefit of U.S. Provisional Patent Application No. 62/959,273, filed Jan. 10, 2020, and incorporates the disclosure of the application by reference.

### BACKGROUND OF THE TECHNOLOGY

Motion sensing devices for personal use, commonly referred to as “activity trackers” or “fitness trackers” are devices for monitoring motion-related metrics such as: steps taken in day, total distance moved, the number of stairs climbed, speed, and estimated calories burned. Over time, a motion sensing device will generate a set of sensor data that can be used to track activity, fitness, geographic location, and other similar data.

Fitness bands and watches are prevalent and commonplace type of fitness tracker. These devices, however, are often made of materials intended for durability and exposure to elements such as perspiration and may not generally be considered to be fashionable or formal in appearance. In addition, many people still spend much of their time in places where fitness-oriented devices are not appropriate or desirable. Furthermore, many consumers already own watches or bracelets and may not have a desire to purchase another similar object that is not attractive or appropriate in certain conditions. Therefore, consumers are left to choose between wearing a fitness band that may not be appropriate for a particular event and wearing a standard object such as a watch which does not contain the functionality to track movement.

### SUMMARY OF THE TECHNOLOGY

A portable universal monitoring device according to various aspects of the present technology includes a portable body housing a plurality of sensor devices. The portable body is configured to be repeatedly attached to and detached from one or more objects such as a wristwatch. The portable body is configured to be discretely positioned on the object to remain unseen without negatively affecting the fit or operation of the object during use. The portable body may be used interchangeably with any number of objects without requiring the objects themselves to be modified.

### BRIEF DESCRIPTION OF THE DRAWINGS

A more complete understanding of the present technology may be derived by referring to the detailed description and claims when considered in connection with the following illustrative figures. In the following figures, like reference numbers refer to similar elements and steps throughout the figures.

FIG. 1 representatively illustrates a rear perspective view of a portable sensor device coupled to a rear facing side of a wristwatch in accordance with an exemplary embodiment of the present technology;

FIG. 2 representatively illustrates a side view of the portable sensor device coupled to a rear facing side of a wristwatch in accordance with an exemplary embodiment of the present technology;

FIG. 3 representatively illustrates a rear view of the portable sensor device coupled to a rear facing side of a wristwatch in accordance with an exemplary embodiment of the present technology;

FIG. 4 representatively illustrates the portable sensor device decoupled from the rear facing side of a wristwatch in accordance with an exemplary embodiment of the present technology;

FIG. 5 representatively illustrates a top perspective view of the portable sensor device in accordance with an exemplary embodiment of the present technology;

FIG. 6 representatively illustrates a bottom perspective view of the portable sensor device in accordance with an exemplary embodiment of the present technology;

FIG. 7 representatively illustrates an exploded view of the components of the portable sensor device in accordance with an exemplary embodiment of the present technology;

FIG. 8 representatively illustrates a top view of the portable sensor device in accordance with an exemplary embodiment of the present technology;

FIG. 9 representatively illustrates a side view of the portable sensor device in accordance with an exemplary embodiment of the present technology;

FIG. 10 representatively illustrates a bottom view of the portable sensor device in accordance with an exemplary embodiment of the present technology;

FIG. 11 representatively illustrates a sync screen of a graphical user interface in accordance with an exemplary embodiment of the present technology;

FIG. 12 representatively illustrates a profile screen of the graphical user interface in accordance with an exemplary embodiment of the present technology;

FIG. 13 representatively illustrates a dashboard screen of the graphical user interface in accordance with an exemplary embodiment of the present technology;

FIG. 14 representatively illustrates a summary screen for a first set of collected sensor data in accordance with an exemplary embodiment of the present technology;

FIG. 15 representatively illustrates a summary screen for a second set of collected sensor data in accordance with an exemplary embodiment of the present technology;

FIG. 16 representatively illustrates a pulse rate screen of the graphical user interface in accordance with an exemplary embodiment of the present technology;

FIG. 17 representatively illustrates a summary screen of caloric activity in accordance with an exemplary embodiment of the present technology;

FIG. 18 representatively illustrates a goals screen of the graphical user interface in accordance with an exemplary embodiment of the present technology;

FIG. 19 representatively illustrates a first exercise tracking screen in accordance with an exemplary embodiment of the present technology;

FIG. 20 representatively illustrates a second exercise tracking screen in accordance with an exemplary embodiment of the present technology; and

FIG. 21 representatively illustrates a sleep summary screen of the graphical user interface in accordance with an exemplary embodiment of the present technology.

Elements and steps in the figures are illustrated for simplicity and clarity and have not necessarily been rendered according to any particular sequence. For example, steps that may be performed concurrently or in a different order are illustrated in the figures to help to improve understanding of embodiments of the present technology.

### DETAILED DESCRIPTION OF EXEMPLARY EMBODIMENTS

The present technology may be described in terms of functional block components and various processing steps. Such functional blocks may be realized by any number of components configured to perform the specified functions and achieve the various results. For example, the present technology may employ various processors, sensors, communication devices, and memory storage devices, which may carry out a variety of operations suited to a specified application or environment. In addition, the present technology may be practiced in conjunction with any number of systems configured for tracking or monitoring personal activity, sensing ambient conditions, collecting biometric data, determining geographic location, and communication between mobile devices, wireless devices, user interfaces, communication systems, open source software applications, and social media platforms, and the system described is merely one exemplary application for the technology. Further, the present technology may employ any number of techniques for collecting data, transferring data, communicating over wireless networks, and electrical charging.

Methods and apparatus for a portable universal monitoring device according to various aspects of the present technology may operate in conjunction with any type of motion sensing system, tracking technology, or vital sign monitoring system. Various representative implementations of the present technology may allow the portable universal monitoring device to be discretely attachable to and detachable from multiple objects or secondary devices so that the portable universal monitoring device may be used across multiple objects without the need to modify the object itself, draw operational power from the object, or be outwardly visible on the object itself. For example, the described technology may be coupled to the back of a first wristwatch during use and then detached or otherwise decoupled from the first wristwatch and coupled to the back of a second wristwatch for continued use allowing the portable universal monitoring device to be worn and used with both wristwatches, or any additional number of wristwatches, according to a given user's preference. More specifically, the user may select to attach the portable universal monitoring device to a first wristwatch worn during the day, then choose to attach the portable universal monitoring device to a second wristwatch worn during a workout, and then choose to attach the portable universal monitoring device to a third wristwatch worn to an evening event. In each instance the user may be able to attach and detach the portable universal monitoring device from a given wristwatch without the use of a tool thereby allowing the user to quickly and easily select the wristwatch, or other secondary device or object, to use with the portable universal monitoring device.

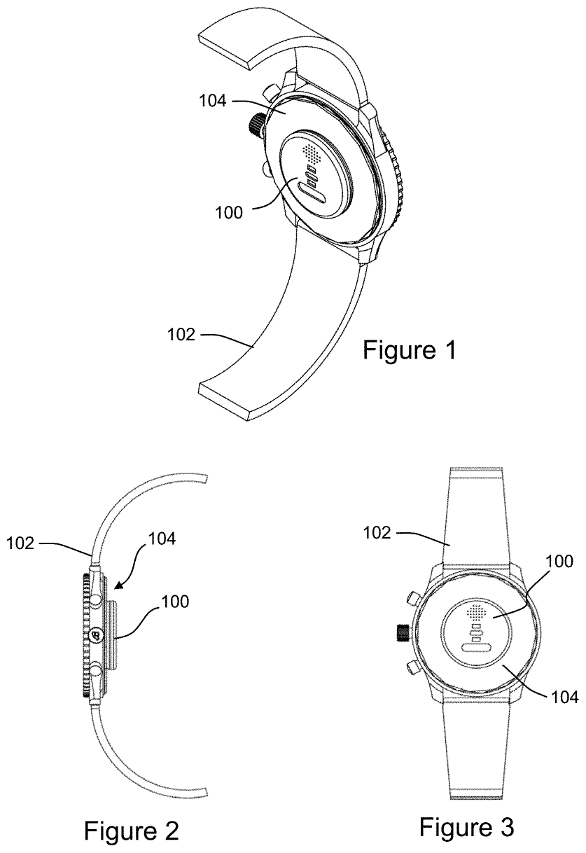

Referring to FIGS. 1-4, the portable universal monitoring device 100 may comprise a size and shape that allows for it to be coupled to an object, such as a wristwatch 102, without deterring from the style of the watch or being visible to other people. In an exemplary embodiment, the portable universal monitoring device 100 may comprise dimensions that are smaller and thinner than the wristwatch 102. For example, in one embodiment, the portable universal monitoring device 100 may comprise a diameter of less than about 3 cm and a thickness of less than about 2 mm to facilitate the discrete placement of the portable universal monitoring device 100 on a rear facing surface 104 back of the wristwatch 102 in such a manner as to be hidden from view when the wristwatch 102 is worn. In this embodiment, the outer diameter of the portable universal monitoring device 100 is less than the outer diameter of the wristwatch 102 and will not protrude outward from the wristwatch 102 when connected to the rear of the wristwatch 102 and worn by the user.

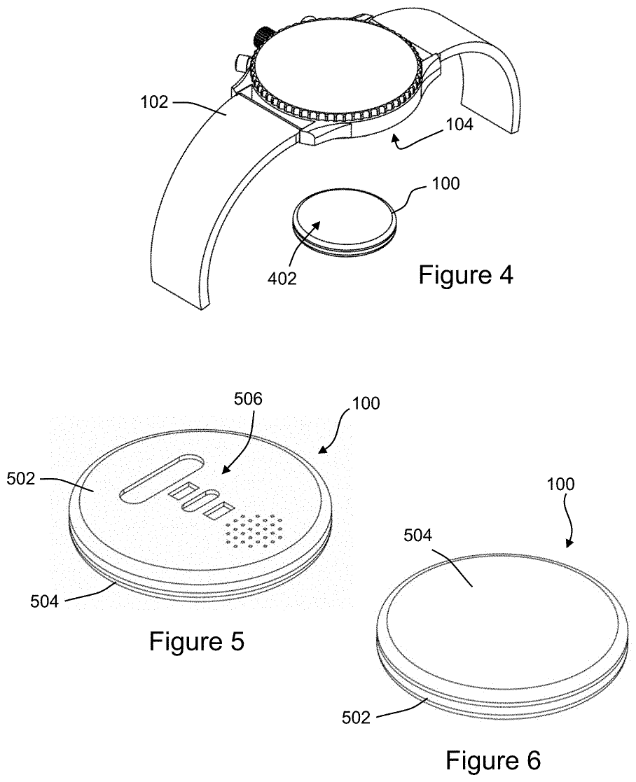

The portable universal monitoring device 100 may be configured to be attached to the object by any suitable method. In one embodiment, and referring now to FIG. 4, a rear facing surface 402 of the portable universal monitoring device 100 may be magnetized in a manner so that it may be attached to the rear facing surface 104 of the wristwatch 102. In another embodiment, the rear facing surface 402 of the portable universal monitoring device 100 may be configured to connect to the rear facing surface 104 of the wristwatch through a negative pressure device such as a suction cup.

In an alternative embodiment, the rear facing surface 104 of the wristwatch 102 may comprise a surface treatment, such as one part of a hook-and-loop fastener, that provides a temporary adhesive connection between the portable universal monitoring device 100 and the rear facing surface 104 of the wristwatch 102 so they may be non-permanently attached together. Alternatively, a temporary adhesive may be applied to the rear facing surface 402 of the portable universal monitoring device 100 to allow the two surfaces to be connected to each other. In yet another embodiment, the portable universal monitoring device 100 may comprise an attachment mechanism having a first part disposed on an exterior surface of the portable universal monitoring device 100 and a second mating part that is attached to the object. The two parts may allow the portable universal monitoring device 100 to be selectively coupled to and decoupled from the object. The user may be able to obtain multiples of the second mating part to allow for attachment to various objects thereby allowing the portable universal monitoring device 100 to be interchangeably used with more than one object or wristwatch 102.

Referring now to FIGS. 5-7, the portable universal monitoring device 100 may comprise a housing made up of a housing face 502 and a housing rear 504 configured to form an interior volume when coupled together. The housing face 502 and the housing rear 504 may be configured to seal off the interior volume from external debris and moisture to provide at least some protection against intrusion. For example, in one embodiment the housing may be moisture resistant to protect against water droplets or light splashing. In an alternative embodiment, the housing may be configured to be water resistant and capable of being submerged in water for several feet.

The housing face 502 may comprise one or more sensor ports 506 configured to provide internal sensors with access to ambient conditions as necessary. For example, a first sensor port 506 may allow ambient light to contact a light sensor, a second sensor port 506 may allow a pressure sensor to detect ambient pressure conditions, while a third sensor port 506 may allow another sensor to detect a user's pulse rate.

The sensor ports 506 may comprise any suitable opening or sensor medium configured to allow a sensor positioned proximate a corresponding sensor port 506 to collect the appropriate data. For example, some sensor ports 506 may comprise an opening in the housing face 502 configured to allow a sensor to detect an ambient condition. Another sensor port 506 may comprise a control button. The sensor ports 506 may also be sealed or partially covered to prevent dust or other contaminants from entering the internal volume of the housing.

The housing face 502 may comprise any suitable material or finish. In one embodiment, an exterior surface of the housing face 502 may comprise a smooth surface. For example, the exterior surface of the housing face 502 may comprise a smooth metallic surface similar to that of the rear facing surface 104 of the wristwatch 102 so as to avoid any discomfort to the user during use.

A microcircuit card 702 may be positioned inside of the internal volume formed by the housing face 502 and the housing rear 504. The microcircuit card 702 may comprise any suitable system or device for tracking and/or collecting data relating to a user's movements or activity, location, and vital signs. The microcircuit card 702 may comprise a main microcontroller, input output ports (I/O ports), a memory device and any other suitable components. For example, in one embodiment, the microcircuit card 702 may comprise a plurality of sensors such as: an accelerometer, gyro, magnetometer, pressure sensor, temperature sensor, step counter, haptic driver, heart rate monitor, blood oximeter, memory device, Bluetooth transmitter/receiver, a micro Global Positioning System (GPS) chip, display device, control buttons, or any other suitable like sensors and components that are configured to collect a set of sensor data.

The microcircuit card 702 may also be responsive to a wireless signal received over a communication network such as Bluetooth®, wireless Ethernet (802.11b), near field communication, a mobile communication network, or a similar technology. For example, the microcircuit card 702 may be responsive to a signal received from a secondary device such as a smart watch or a separate remote device such as a mobile phone or tablet computer and communicate or otherwise transfer collected sensor data to the remote device.

The microcircuit card 702 may be powered by a rechargeable battery 704 positioned within the interior volume and electrically coupled to the microcircuit card 702. In some embodiments, the battery 704 may comprise any thin film, flexible, or printed battery cell that may allow for safe transmission of power to the electronic components without risk of fire. For example, the battery 704 may comprise advanced lithium-ion batteries, solid-state batteries, micro-batteries, stretchable batteries, thin flexible supercapacitors, or a manganese dioxide-based battery. The battery 704 may be configured to be charged by an inductive system to avoid any need for a wired connection and to help maintain a low profile of the portable universal monitoring device 100. For example, in one embodiment, a charging coil 706 may be coupled to the battery 704 to allow for wireless inductive charging.

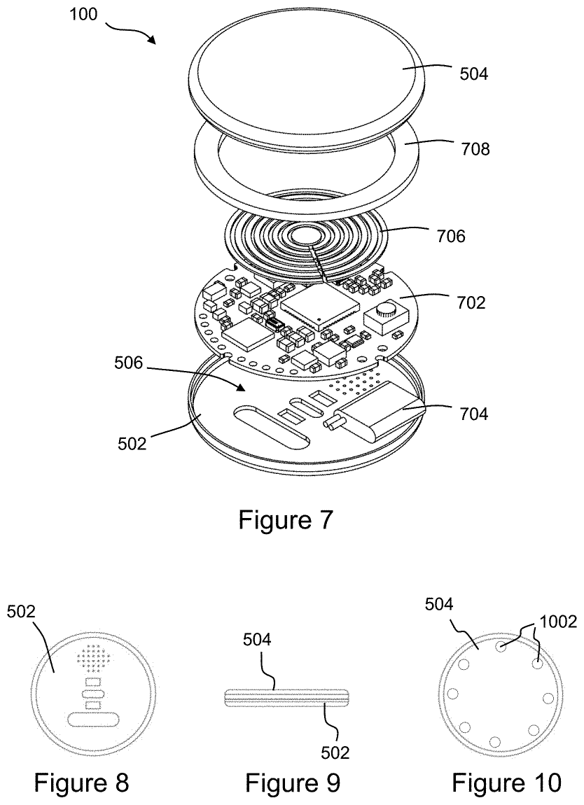

The housing rear 504 is configured to be positioned adjacent to the object when the portable universal monitoring device 100 and the object are coupled or otherwise attached to each other. In one embodiment, the housing rear 504 may comprise a metallic body that has been magnetized such that it can be attached to a metallic surface of the object. For example, the entire metallic body itself may be magnetized or a plurality of magnets 1002 may be embedded into the surface of the housing rear 504 (See FIG. 10). In a second embodiment, and referring again to FIG. 7, a ring magnet 708 may be positioned within the housing and located immediately adjacent to an interior surface of the housing rear 504. The ring magnet 708 may comprise a sufficiently strong magnetic force that it can securely couple the portable universal monitoring device 100 to another object, such as the rear facing surface 104 of the wristwatch 102. In a third embodiment, a plurality of small magnets may be positioned along the interior surface of the housing rear 504 such that the collective magnetic force created by the individual magnets is sufficient to secure the portable universal monitoring device 100 to the object.

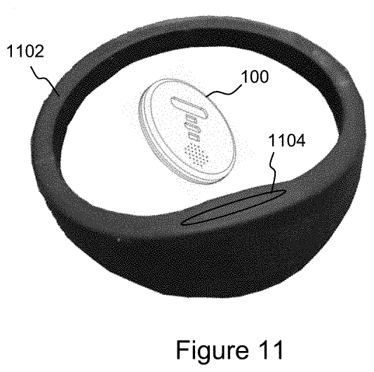

Referring now to FIG. 11, the size of the portable universal monitoring device 100 may also allow it to be connected to and used with non-metallic objects. In one embodiment, the portable universal monitoring device 100 may be configured to be placed inside of a bracelet 1102 made of rubber, plastic, or some other polymer suitably configured to use during exercise. For example, the bracelet 1102 may be made from a material that is resistant to perspiration, water, and abrasion.

The bracelet 1102 may comprise a slot 1104 disposed along a surface of the bracelet that is configured to receive the portable universal monitoring device 100 into an inner portion of the bracelet 1102. The slot 1104 may be disposed along any surface of the bracelet 1102 such as a rear facing surface or an externally facing surface such as a side or front of the bracelet 1102. The inner portion of the bracelet may also be configured with a metal surface that more securely couples to the housing rear 504 to provide a more secure connection between the bracelet 1102 and the portable universal monitoring device 100.

The portable universal monitoring device 100 may be configured to communicate with an application software program or “App” installed on a separate remote device, such as: a smart phone; tablet computer; or other personal computing device. The App may allow the user to review, process, or analyze sensor data collected by the plurality of sensor devices on the portable universal monitoring device 100. The App may comprise any suitable system of interface that allows the user to view or otherwise access the collected data. The App may be installed locally on the separate remote device or it may be installed as a cloud-based application that gives the user multiple ways of accessing the collected data. For example, the user may be able to access a virtual storefront that provides access to various software applications for a particular platform or operating system such as iOS or Android® through the internet by using the remote device to connect to an application delivery system via the internet. The user may then select the App and have it delivered to their remote device over the internet.

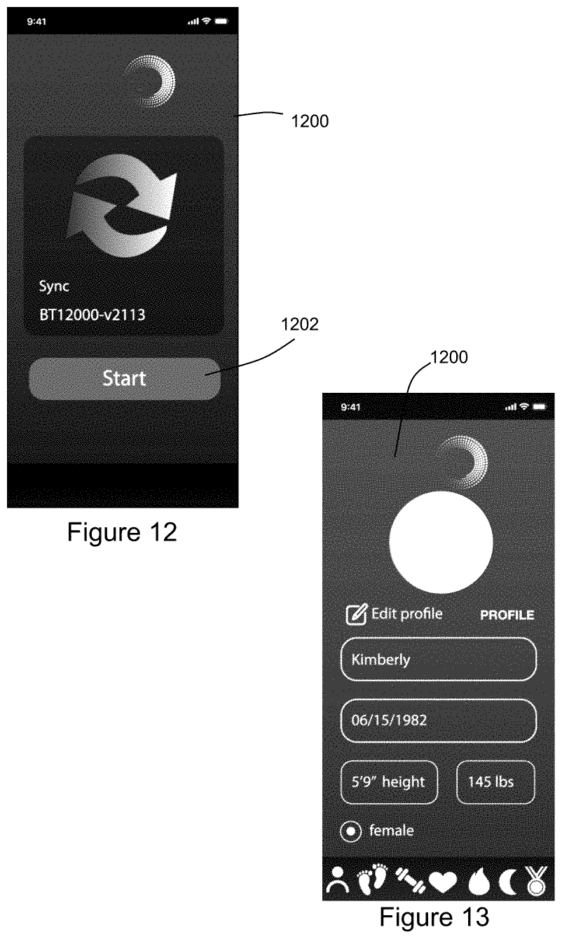

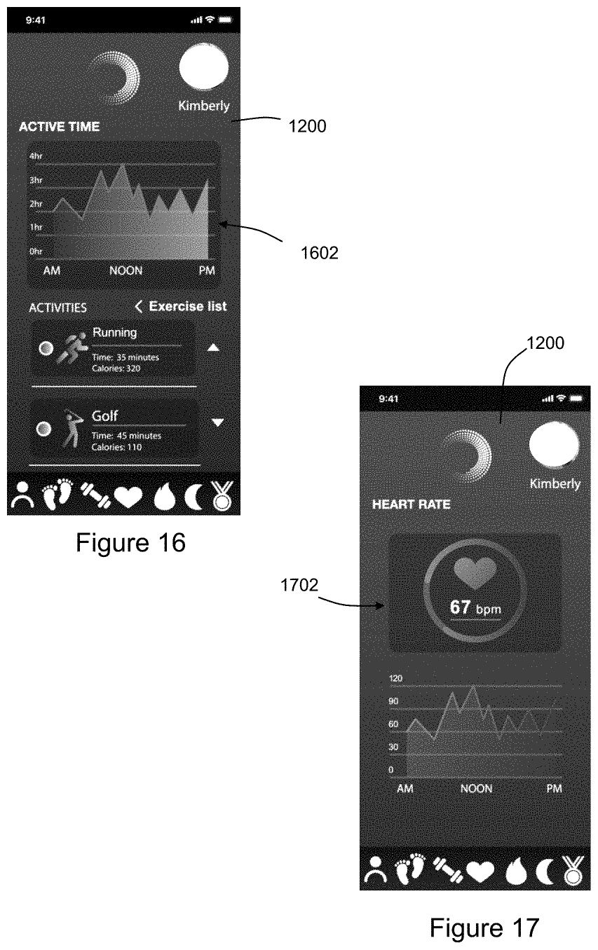

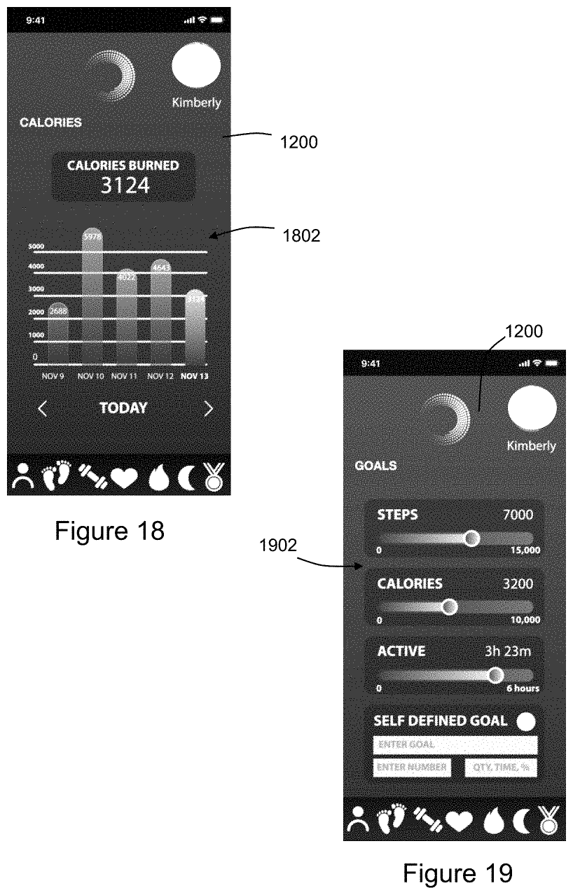

The App may comprise a set of computer implemented instructions for enabling the remote device to communicate with the portable universal monitoring device 100. Referring now to FIGS. 12-22, the App may be configured to display a graphical user interface (GUI) 1200 on a screen of the remote device. The GUI 1200 may be adapted to present the user with various data screens for allowing access to specific types of sensor data collected by the portable universal monitoring device 100. For example, and with particular reference now to FIG. 12, when the App is opened on the remote device and detects the portable universal monitoring device 100 the user may be prompted to upload recently captured sensor data from the portable universal monitoring device 100 to the remote device. This syncing process may also be configured to be automatic allowing for sensor data to be transferred from the portable universal monitoring device 100 to the remote device at regular intervals, in real-time, or while the App is active.

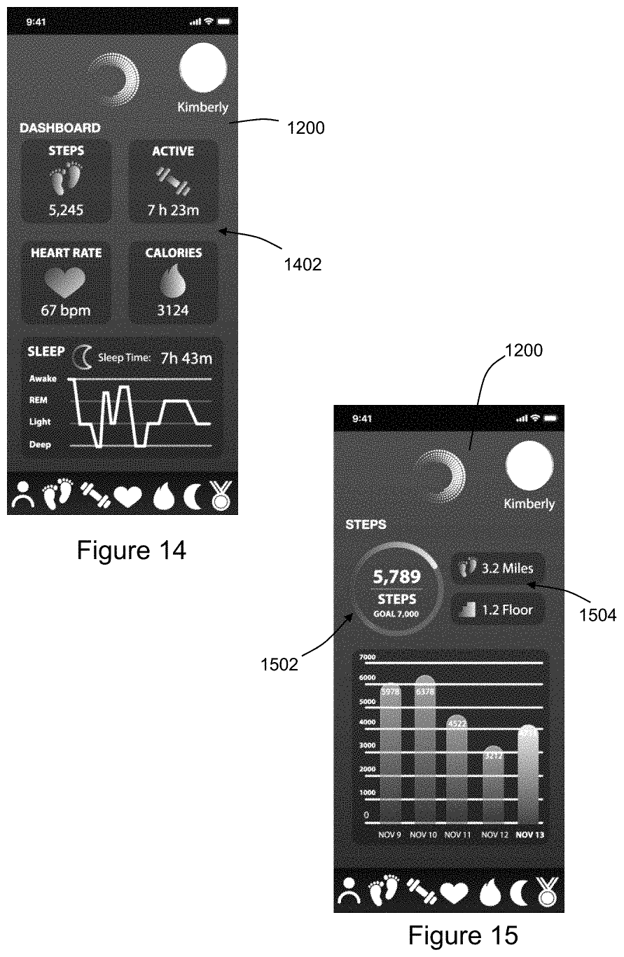

The App may also allow each user to create a personal profile (see FIG. 13). This profile may then be associated with collected sensor data to allow the user to track or review collected sensor data. For example, referring now to FIG. 14, the App may display a general dashboard to the user with a summary 1402 of collected sensor data such as the number of steps taken over a given period of time, the number of minutes of detected movement or activity, estimated calories burned over a given period of time, and current biometric data such as a pulse rate or current blood oxygen level. The sensor data or information shown on the dashboard may be preselected or customizable such that the user can select the sensor data presented on the dashboard.

The App may also allow the user to select any of the displayed data field on the dashboard to obtain a more detailed view of the sensor data collected. For example, and referring now to FIGS. 15-18, in response to the user pressing on the steps icon on the dashboard, the App may present a new screen view (see FIG. 15) to provide a detailed view 1502 of the total number of steps taken and the calculated distance 1504 covered by the steps. Similarly, if the user selects any of the other fields on the dashboard the App may display additional screens detailing active time 1602, pulse rate 1702, and caloric data 1802.

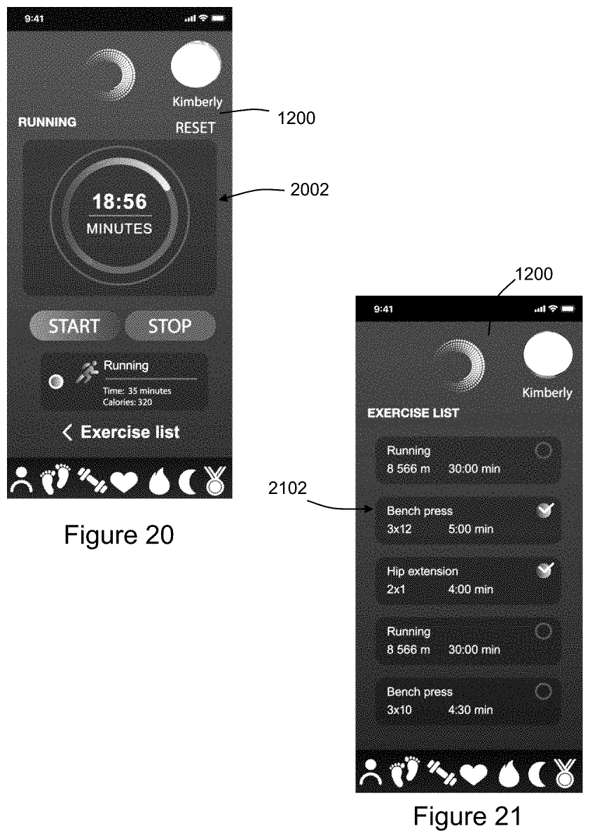

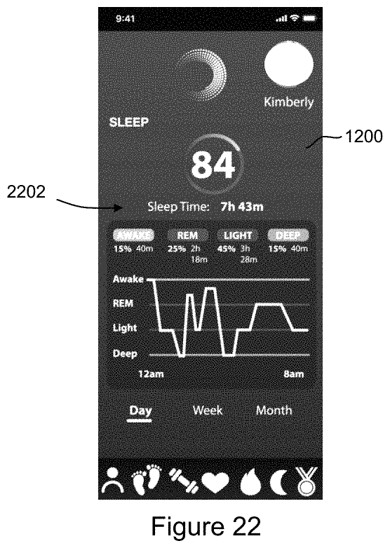

Referring now to FIG. 19, the App may also be configured to allow the user to set desired goals corresponding to specific sensor data that is collected by portable universal monitoring device 100. For example, the user may be able to select a set of goals 1902 from a list of available options which the App may then track. The App may also allow the user to track specific discrete elements such as a workout or exercise. Referring now to FIGS. 20 and 21, the App may allow the user to select from a predetermined set 2002 of exercises and then track the user's performance. For example, if the user selects running, the App may collect sensor data from the portable universal monitoring device 100 relating to speed, geographic location, elevation changes, heart rate, body temperature, and any other suitable factors or elements that the portable universal monitoring device 100 has a sensor for. At the end of the exercise, the App may present the collected sensor data to the user as a summary 2102 of the workout.

Referring now to FIG. 22, the App may also be configured to monitor a user's sleep profile by monitoring sleep factors 2202 such as heart rate, length of time spent not moving, and ambient conditions such as room temperature. A sleep screen may display the collected sleep factors 2202 to the user.

The description and figures are to be regarded in an illustrative manner, rather than a restrictive one and all such modifications are intended to be included within the scope of the present technology. Accordingly, the scope of the technology should be determined by the generic embodiments described and their legal equivalents rather than by merely the specific examples described above. For example, the components and/or elements recited in any apparatus embodiment may be assembled or otherwise operationally configured in a variety of permutations to produce substantially the same result as the present technology and are accordingly not limited to the specific configuration recited in the specific examples.

As used herein, the terms “comprises,” “comprising,” or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present technology, in addition to those not specifically recited, may be varied or otherwise particularly adapted to specific environments, manufacturing specifications, design parameters or other operating requirements without departing from the general principles of the same.

The present technology has been described above with reference to exemplary embodiments. However, changes and modifications may be made to the exemplary embodiments without departing from the scope of the present technology. These and other changes or modifications are intended to be included within the scope of the present technology, as expressed in the following claims.

1. A portable universal sensor device, comprising:
a housing face;
a housing rear coupled to the housing face to form an internal housing volume;
a microcircuit card disposed within the internal housing volume comprising a plurality of connected sensor devices configured to collect a set of sensor data; and
a ring magnet disposed within the internal housing volume immediately adjacent to an interior facing surface of the housing rear.

2. A portable universal sensor device according to claim 1, further comprising a plurality of sensor ports disposed on the housing face.

3. A portable universal sensor device according to claim 2, wherein each housing port comprises an opening in the housing face that corresponds to a location of a sensor device connected to the microcircuit card.

4. A portable universal sensor device according to claim 1, further comprising:
a battery electrically coupled to and configured to power the microcircuit card; and
a charging coil electrically coupled to and configured to recharge the battery.

5. A portable universal sensor device according to claim 1, wherein the microcircuit card is configured to transmit the collected sensor data to a remote device over a wireless communication network.

6. A portable universal sensor device according to claim 5, further comprising an application software system configured to be installed on the remote device and adapted to receive and process the transmitted sensor data for display on the remote device.

7. A portable universal sensor device according to claim 1, further comprising a polymer bracelet configured to receive the coupled housing face and housing rear within an inner portion of the bracelet.

8. A portable universal sensor device according to claim 7, further comprising a metallic surface disposed along a surface of the inner portion of the bracelet.

9. A portable universal sensor device for use with a wristwatch, comprising:
a housing face;
a housing rear coupled to the housing face to form an internal housing volume, wherein the housing rear is configured to be detachably connected to a rear surface of the wristwatch; and
a microcircuit card disposed within the internal housing volume comprising a plurality of connected sensor devices configured to collect a set of sensor data.

10. A portable universal sensor device according to claim 9, further comprising a plurality of sensor ports disposed on the housing face.

11. A portable universal sensor device according to claim 10, wherein each housing port comprises an opening in the housing face and corresponds to a location of a sensor device connected to the microcircuit card.

12. A portable universal sensor device according to claim 9, further comprising:
a battery electrically coupled to and configured to power the microcircuit card; and
a charging coil electrically coupled to and configured to recharge the battery.

13. A portable universal sensor device according to claim 9, wherein the microcircuit card is configured to transmit the collected sensor data to a remote device over a wireless communication network.

14. A portable universal sensor device according to claim 13, further comprising an application software system configured to be installed on the remote device and adapted to receive and process the transmitted sensor data for display on the remote device.

15. A portable universal sensor device according to claim 9, further comprising a ring magnet disposed within the internal housing volume immediately adjacent to an interior facing surface of the housing rear.

16. A portable universal sensor device according to claim 9, wherein at least a portion of an exterior surface of the housing rear is magnetized.

17. A portable universal sensor device according to claim 16, further comprising at least one magnet embedded in the housing rear.

18. A portable universal sensor device according to claim 9, further comprising a polymer bracelet configured to receive the coupled housing face and housing rear within an inner portion of the bracelet.

19. A portable universal sensor device according to claim 18, further comprising a metallic surface disposed along a surface of the inner portion of the bracelet.
